(19) United States
(12) Patent Application Publication
(10) Pub. No.: US 2026/0102178 A1
(43) Pub. Date: Apr. 16, 2026

(54) END EFFECTOR KNIFE WITH RETENTION PLATEAU

(71) Applicant: CILAG GMBH INTERNATIONAL, Zug (CH)

(72) Inventors: Jeff CLARK, Cincinnati, OH (US); Guowei John ZHANG, Cincinnati, OH (US); James WILSON, Cincinnati, OH (US); Steve SMOLIK, Cincinnati, OH (US)

(73) Assignee: CILAG GMBH INTERNATIONAL, Zug (CH)

(21) Appl. No.: 18/915,982

(22) Filed: Oct. 15, 2024

(51) Int. Cl. A61B 17/295; A61B 34/00; A61B 34/30

(52) U.S. Cl. CPC A61B 17/295; A61B 34/71; A61B 2034/302

(57) ABSTRACT

An end effector for a surgical tool includes opposing first and second jaws, and a knife slot defined in one or both of the first and second jaws, and a knife extendable through the knife slot and operatively couplable to a distal end of a knife rod, the knife providing a body having a leading end, a trailing end opposite the leading end, and opposing first and second side surfaces extending between the leading and trailing ends, a central slot defined in the body and extending from the trailing end, the central slot being sized to receive the distal end of the knife rod, and a retention plateau defined on the first side surface and laterally protruding from the first side surface, wherein the retention plateau circumscribes the central slot.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

Some end effectors also include a knife that is able to be advanced and retracted between opposing jaws to cut or sever tissue grasped between the opposing jaws. Improvements to the knife design affecting the guidance and retention of the knife on a knife rod are desirable to improve the efficiency of the end effector and any procedures undertaken with the end effector.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIG. 5 is another enlarged isometric view of the distal end of the surgical tool of FIG. 2, according to one or more embodiments of the present disclosure.

FIG. 6 is an enlarged side view of an end effector, according to one or more embodiments of the present disclosure.

FIG. 7 is an isometric view of the knife of FIGS. 5 and 6, in accordance with the principles of the present disclosure.

FIG. 8 is another enlarged, isometric view of the knife of FIGS. 5 and 6, according to one or more additional embodiments of the present disclosure.

FIGS. 9A and 9B are enlarged front (end) views of the knife of FIGS. 5 and 6, according to various embodiments of the present disclosure.

FIGS. 10A and 10B are enlarged side views of alternate examples of the knife of FIGS. 5 and 6, according to one or more additional embodiments of the present disclosure.

### DETAILED DESCRIPTION

The present disclosure is related to surgical tools and, more particularly, to end effectors that include a knife and designs for improving knife attachment and functionality.

Embodiments discussed herein describe an end effector for a surgical tool, where the end effector includes opposing first and second jaws, and a knife slot defined in one or both of the first and second jaws. A knife is extendable through the knife slot and may be operatively coupled to the distal end of a knife rod at a retention plateau defined on at least one side surface of the knife. More specifically, the retention plateau laterally protrudes from at least one side surface of the knife and defines a transitional edge between the retention plateau and the side surface of the knife. In at least one embodiment, the knife and the retention plateau define a central slot, such that the knife rod is receivable within the central slot to operatively couple the knife rod and the knife. The central slot may define one or more notches protruding from an internal surface of the central slot to aid in locating and securing the knife rod within the central slot.

In some embodiments, a retention plateau is defined on opposing side surfaces of the knife such that a total thickness of the knife and the retention plateaus is evenly distributed on either side surface of the knife. In these embodiments, the knife rod may be centrally located within the central slot of the retention plateau, thus aligning a centerline of the knife rod with the knife. In alternate embodiments, however, a single retention plateau may be provided on one side surface of the knife, such that the centerline of the knife rod is laterally offset from the center of the knife (e.g., the cutting edge). In these varying embodiments, the total thickness of the knife and retention plateaus may be equivalent to provide an equivalent retention force between the embodiments. The varying alignments between the centerline of the knife rod and the knife may provide advantages based upon the desired application of the knife and surgical tool.

The leading edge of the retention plateau can exhibit varying shapes and geometries based upon a desired application and parameters for the end effector and surgical tool. In some embodiments, the leading edge of the retention plateau can provide a bullnose shape with a rounded tip, such that no angular surfaces are present on the retention plateau to prevent snagging or cutting. In further embodiments, the leading edge of the retention plateau can exhibit an angularly tapered or conical shape that is brought to a rounded point towards the cutting edge of the knife. The angularly tapered shape can provide additional guidance for cutting through tissue, as the tissue may be urged apart following cutting as it travels along the leading edge. The shape or geometry of the leading edge may be further chosen to guide the knife through a knife slot of the end effector, such that the retention plateau enables proper seating and travel therein. Further, some embodiments disclosed herein may provide varying numbers and locations of notches protruding from the internal surface of the central slot. The notches may be provided to enhance locating and mating between the knife and the knife rod prior to operative coupling therebetween. As such, various embodiments can provide these varying designs to enhance retention or reduce slip within the central slot, or to save valuable real estate within the knife through reduced numbers of notches.

The varying embodiments and designs disclosed herein may maximize the retention forces between the knife rod and the knife of an end effector of a surgical tool. The increased retention forces may reduce the risk of damage at the common point of failure at the connection between the knife rod and the knife. The disclosed embodiments may thus strengthen this connection without reducing cutting efficiency of the knife through the provided retention plateau.

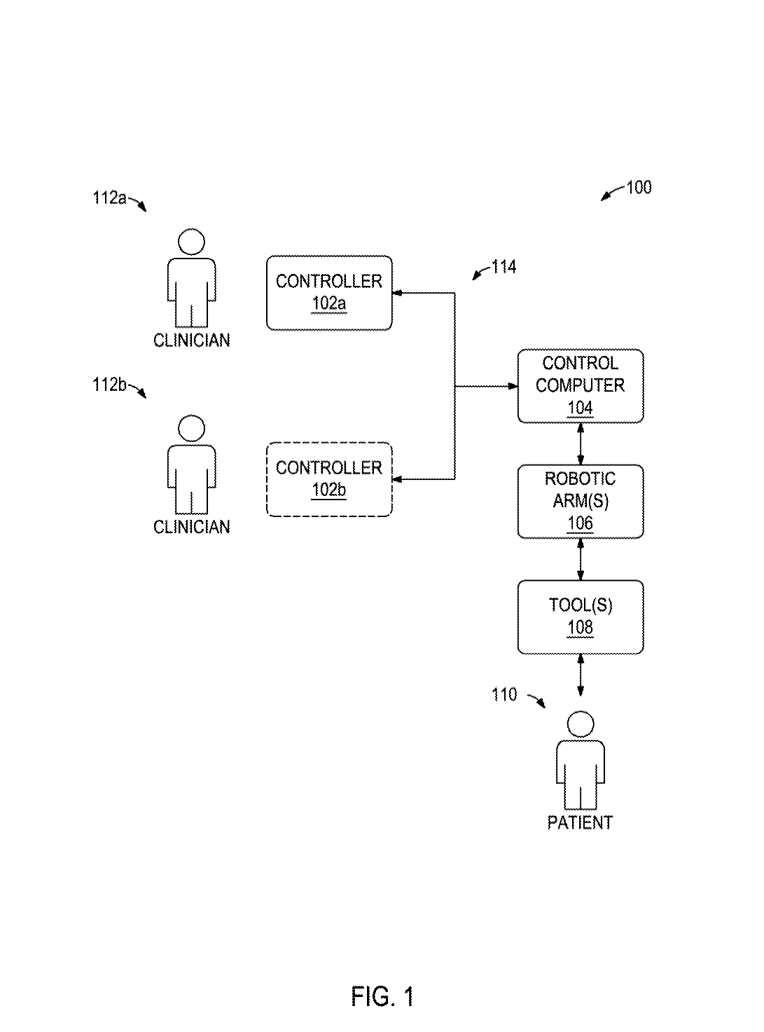

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

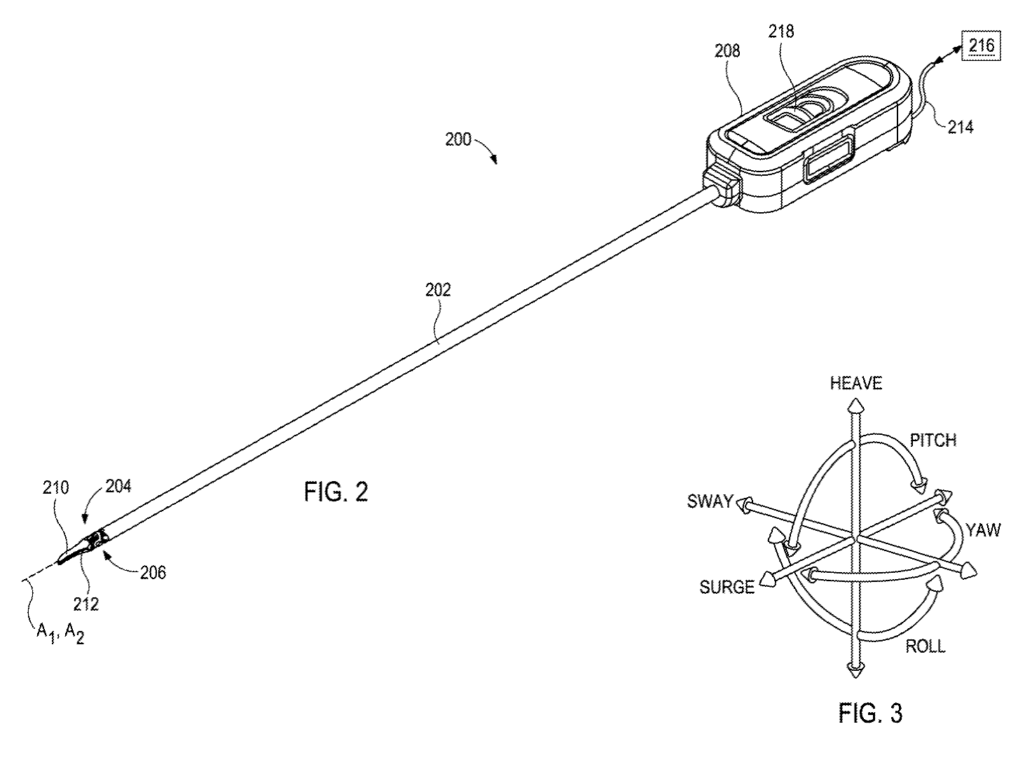

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the system 100 of FIG. 1. Accordingly, the surgical tool 200 may be designed to be releasably coupled to a tool driver included in the system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A₁. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises a combination tissue grasper and vessel sealer that include opposing first (upper) and second (lower) jaws 210, 212 configured to move (articulate) between open and closed positions. As will be appreciated, however, the opposing jaws 210, 212 may alternatively form part of other types of end effectors such as, but not limited to, a surgical scissors, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator 216 that supplies energy, such as electrical energy (e.g., radio frequency energy), ultrasonic energy, microwave energy, heat energy, or any combination thereof, to the surgical tool 200 and, more particularly, to the end effector 204. Accordingly, the generator 216 may comprise a radio frequency (RF) source, an ultrasonic source, a direct current source, and/or any other suitable type of electrical energy source that may be activated independently or simultaneously.

In applications where the surgical tool 200 is configured for bipolar operation, the power cable 214 will include a supply conductor and a return conductor. Current can be supplied from the generator 216 to an active (or source) electrode located at the end effector 204 via the supply conductor, and current can flow back to the generator 216 via a return electrode located at the end effector 204 via the return conductor. In the case of a bipolar grasper with opposing jaws, for example, the jaws serve as the electrodes where the proximal end of the jaws are isolated from one another and the inner surface of the jaws (i.e., the area of the jaws that grasp tissue) apply the current in a controlled path through the tissue. In applications where the surgical tool 200 is configured for monopolar operation, the generator 216 transmits current through a supply conductor to an active electrode located at the end effector 204, and current is returned (dissipated) through a return electrode (e.g., a grounding pad) separately coupled to a patient's body.

The surgical tool 200 may further include a manual release switch 218 that may be manually actuated by a user (e.g., a surgeon) to open the jaws 210, 212. The release switch 218 is movably positioned on the drive housing 208, and a user is able to manually move (slide) the release switch 218 from a disengaged position, as shown, to an engaged position. In the disengaged position, the surgical tool 200 is able to operate as normal. As the release switch 218 moves to the engaged position, however, various internal component parts of the drive housing 208 are simultaneously moved, thereby resulting in the jaws 210, 212 opening, which might prove beneficial for a variety of reasons. In some applications, for example, the release switch 218 may be moved in the event of an electrical disruption that renders the surgical tool 200 inoperable. In such applications, the user would be able to manually open the jaws 210, 212 and thereby release any grasped tissue and remove the surgical tool 200. In other applications, the release switch 218 may be actuated (enabled) to open the jaws 210, 212 in preparation for cleaning and/or sterilization of the surgical tool 200. In some applications, the surgical tool 200 is first decoupled from the robotic manipulator and the associated motors, following which the user can actuate the manual release switch 218 to move the associated inputs and drive the cables once the motors are disengaged.

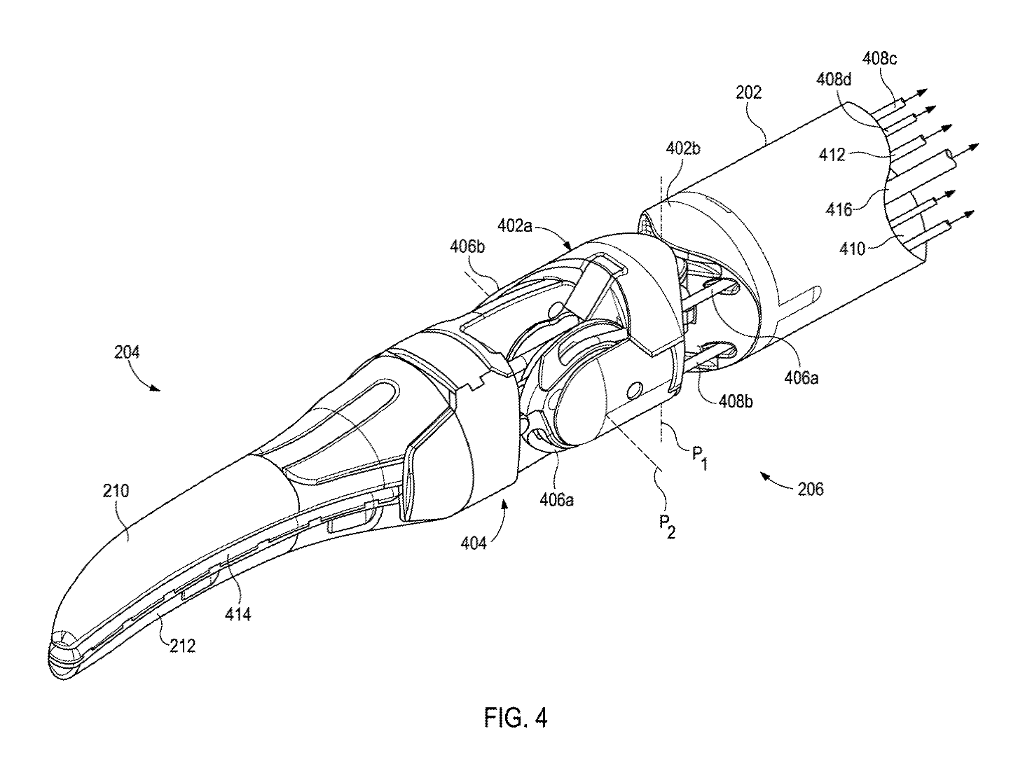

FIG. 4 is an enlarged isometric view of the distal end of the surgical tool 200. More specifically, FIG. 4 depicts an enlarged view of the end effector 204 and the wrist 206, with the jaws 210, 212 of the end effector 204 in the closed position. The wrist 206 operatively couples the end effector 204 to the shaft 202. In some embodiments, however, a shaft adapter may be directly coupled to the wrist 206 and otherwise interpose the shaft 202 and the wrist 206. Accordingly, the wrist 206 may be operatively coupled to the shaft 202 either through a direct coupling engagement where the wrist 206 is directly coupled to the distal end of the shaft 202, or an indirect coupling engagement where a shaft adapter interposes the wrist 206 and the distal end of the shaft 202. As used herein, the term “operatively couple” refers to a direct or indirect coupling engagement between two components.

To operatively couple the end effector 204 to the shaft 202, the wrist 206 includes a first or “distal” clevis 402a and a second or “proximal” clevis 402b. The clevises 402a,b are alternatively referred to as “articulation joints” of the wrist 206 and extend from the shaft 202, or alternatively a shaft adapter. The clevises 402a,b are operatively coupled to facilitate articulation of the wrist 206 relative to the shaft 202. As illustrated, the wrist 206 also includes a linkage 404 arranged distal to the distal clevis 402a and operatively mounted to the jaws 210, 212.

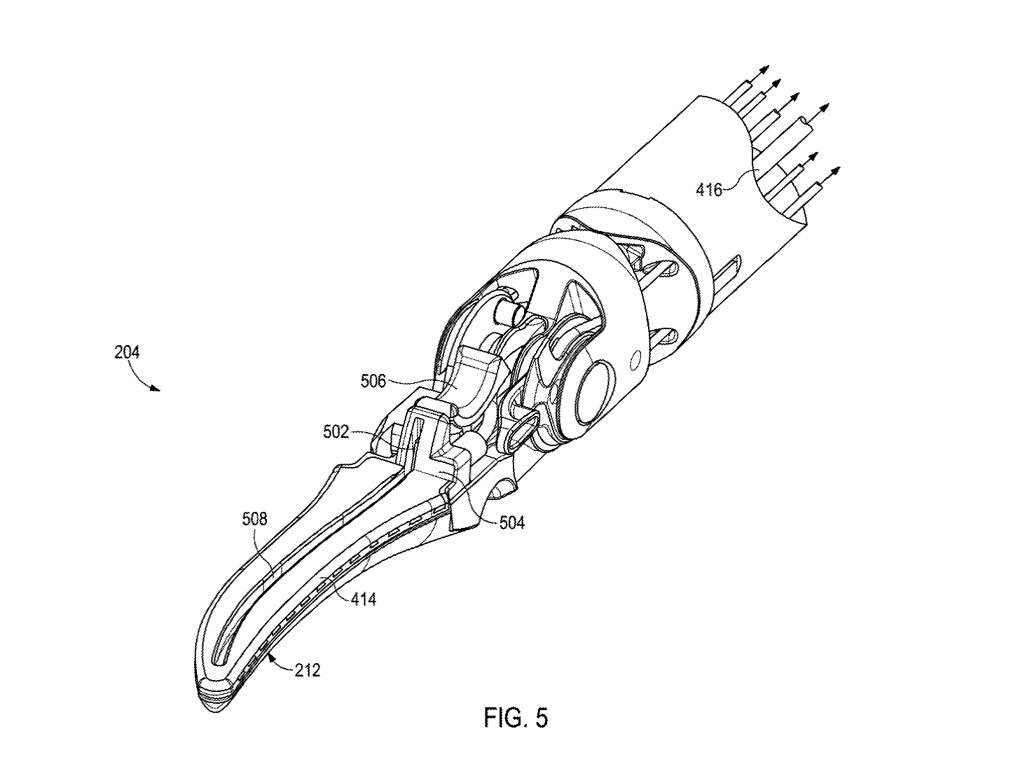

As illustrated, the proximal end of the distal clevis 402a may be rotatably mounted or pivotably coupled to the proximal clevis 402b at a first pivot axis P₁ of the wrist 206. In some embodiments, an axle may extend through the first pivot axis P₁and the distal and proximal clevises 402a,b may be rotatably coupled via the axle. In other embodiments, however, such as is depicted in FIG. 5, the distal and proximal clevises 402a,b may be engaged in rolling contact, such as via an intermeshed gear relationship that allows the clevises 402a,b to rotate relative to each other similar to a rolling joint.

First and second pulleys 406a and 406b may be rotatably mounted to the distal end of the distal clevis 402a at a second pivot axis P₂ of the wrist 206. The linkage 404 may be arranged distal to the second pivot axis P₂ and operatively mounted to the jaws 210, 212. The first pivot axis P₁ is substantially perpendicular (orthogonal) to the longitudinal axis A₁ of the shaft 202, and the second pivot axis P₂ is substantially perpendicular (orthogonal) to both the longitudinal axis A₁ and the first pivot axis P₁. Movement of the end effector 204 about the first pivot axis P₁ provides “yaw” articulation of the wrist 206, and movement about the second pivot axis P₂ provides “pitch” articulation of the wrist 206.

A plurality of drive cables, shown as drive cables 408a, 408b, 408c, and 408d, extend longitudinally within a lumen 410 defined by the shaft 202 (or a shaft adaptor) and extend at least partially through the wrist 206. The drive cables 408a-d may form part of the cable driven motion system housed within the drive housing 208 (FIG. 2), and may comprise cables, bands, lines, cords, wires, woven wires, ropes, strings, twisted strings, elongate members, belts, shafts, flexible shafts, knife rods, or any combination thereof. The drive cables 408a-d can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 408a-d are depicted in FIG. 5, more or less than four may be employed, without departing from the scope of the disclosure.

The drive cables 408a-d extend proximally from the end effector 204 and the wrist 206 toward the drive housing 208 (FIG. 2) where they are operatively coupled to various actuation mechanisms or devices that facilitate longitudinal movement (translation) of the drive cables 408a-d within the lumen 410. Selective actuation of the drive cables 408a-d applies tension (i.e., pull force) to the given drive cable 408a-d in the proximal direction, which urges the given drive cable 408a-d to translate longitudinally within the lumen 410.

In the illustrated embodiment, the drive cables 408a-d each extend longitudinally through the proximal clevis 402b. The distal end of each drive cable 408a-d terminates at the first or second pulleys 406a,b, thus operatively coupling each drive cable 408a-d to the end effector 204. In some embodiments, the distal ends of the first and second drive cables 408a,b may be coupled to each other and terminate at the first pulley 406a, and the distal ends of the third and fourth drive cables 408c,d may be coupled to each other and terminate at the second pulley 406b. In at least one embodiment, the distal ends of the first and second drive cables 408a,b and the distal ends of the third and fourth drive cables 408c,d may each be coupled together at corresponding ball crimps (not shown) mounted to the first and second pulleys 406a,b, respectively.

In at least one embodiment, the drive cables 408a-d may operate “antagonistically”. More specifically, when the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa. Antagonistic operation of the drive cables 408a-d can open or close the jaws 210, 212 and can further cause the end effector 204 to articulate at the wrist 206. More specifically, selective actuation of the drive cables 408a-d in known configurations or coordination can cause the end effector 204 to articulate about one or both of the pivot axes P₁, P₂, thus facilitating articulation of the end effector 204 in both pitch and yaw directions. Moreover, selective actuation of the drive cables 408a-d in other known configurations or coordination will cause the jaws 210, 212 to open or close. Antagonistic operation of the drive cables 408a-d advantageously reduces the number of cables required to provide full wrist 206 motion, and also helps eliminate slack in the drive cables 408a-d, which results in more precise motion of the end effector 204.

In the illustrated embodiment, the end effector 204 is able to articulate (move) in pitch about the second or “pitch” pivot axis P₂, which is located near the distal end of the wrist 206. Thus, the jaws 210, 212 open and close in the direction of pitch. In other embodiments, however, the wrist 206 may alternatively be configured such that the second pivot axis P₂ facilitates yaw articulation of the jaws 210, 212, without departing from the scope of the disclosure.

In some embodiments, an electrical conductor 412 may also extend longitudinally within the lumen 410, through the wrist 206, and terminate at an electrode 414 to supply electrical energy to the end effector 204. In some embodiments, the electrical conductor 412 may comprise a wire, but may alternatively comprise a rigid or semi-rigid shaft, rod, or strip (ribbon) made of a conductive material. The electrical conductor 412 may be entirely or partially covered with an insulative covering (overmold) made of a non-conductive material. Using the electrical conductor 412 and the electrode 414, the end effector 204 may be configured for monopolar or bipolar RF operation.

In the illustrated embodiment, the end effector 204 comprises a combination tissue grasper and vessel sealer that includes a knife (not visible), alternately referred to as a “cutting element” or “blade.” The knife is aligned with and configured to traverse a guide track or “knife slot” (not visible) defined longitudinally in one or both of the upper and lower jaws 210, 212. The knife may be operatively coupled to the distal end of a knife rod 416 (alternately referred to as “drive rod,” “actuation rod,” or “push rod”) that extends longitudinally within the lumen 410 and passes through the wrist 206. Longitudinal movement (translation) of the knife rod 416 correspondingly moves the knife within the knife slot(s). Similar to the drive cables 408a-d, the knife rod 416 may form part of the actuation systems housed within the drive housing 208 (FIG. 2). Selective actuation of a corresponding drive input will cause the knife rod 416 to move distally or proximally within the lumen 410, and correspondingly move the knife in the same longitudinal direction.

The knife rod 416 may comprise a rigid or semi rigid elongate member, such as a rod or shaft (e.g., a hypotube, a hollow rod, a solid rod, etc.), a wire, a ribbon, a push cable, or any combination thereof. The knife rod 416 can be made from a variety of materials including, but not limited to, metal (e.g., tungsten, nitinol, stainless steel, etc.), a polymer, or a composite material. The knife rod 416 may have a circular cross-section, but may alternatively exhibit a polygonal cross-section without departing from the scope of the disclosure.

FIG. 5 is another enlarged isometric view of the end effector 204, according to one or more embodiments of the present disclosure. The upper jaw 210 (FIG. 2 and FIG. 5) is omitted from FIG. 5 to enable viewing of various internal features of the end effector 204.

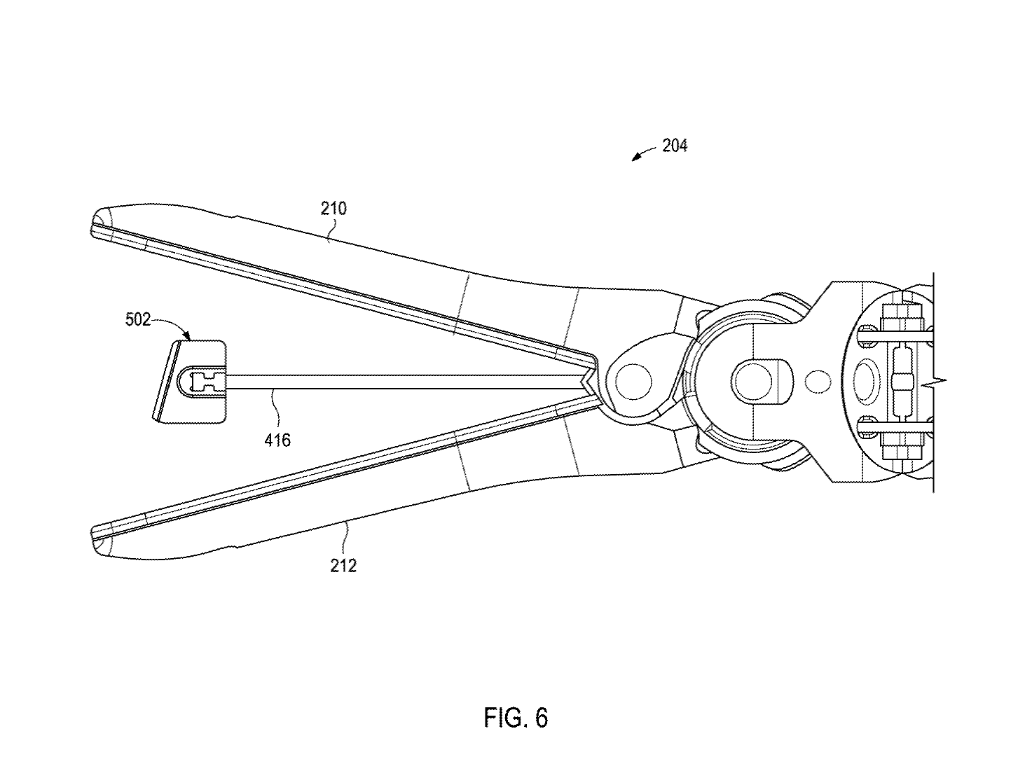

In the illustrated embodiment, a knife 502 (mostly occluded) is shown received within a portion of the electrode 414 of the lower jaw 212 and, more particularly, within a portion of an insulator 504 coupled to the electrode 414. In its retracted position, as shown in FIG. 6, the knife 502 may also be partially received within a knife housing 506 (alternately referred to as a “distal wedge” or “knife garage”) mounted to the end effector 204 between the upper and lower jaws 210, 212. The lower jaw 212 provides or otherwise defines a knife slot 508 (alternately referred to as a “knife track” or “guide track”)) through which the knife 502 may traverse upon distal actuation of the knife rod 416. While the knife slot 508 is shown provided in the lower jaw 212, in some embodiments, the knife slot 508 may be cooperatively defined by both the upper and lower jaws 210, 212.

The knife housing 506 defines a central passageway through which the knife rod 416 is able to extend to move the knife 502 along the knife slot 508. Upon firing the end effector 204, the knife rod 416 is moved (urged) distally, which correspondingly moves the knife 502 out of the knife housing 506 and into the knife slot 508. After firing is complete, the knife rod 416 is retracted proximally, which pulls the knife 502 proximally and back into the knife housing 506 until it is desired to again fire the end effector 204.

### Advanced Bi-Polar Knife with Improved Retention

FIG. 6 is an enlarged side view of the end effector 204, according to one or more embodiments. As illustrated, the end effector 204 includes the upper and lower jaws 210, 212, and the knife 502 is operatively coupled to the distal end of the knife rod 416, which can be longitudinally translated distally and proximally to correspondingly move the knife 502 distally and proximally.

In some embodiments, the knife rod 416 may comprise a solid shaft, but may alternatively comprise a tube or tubular structure. Moreover, the knife rod 416 may be made of a variety of flexible materials including, but not limited to, a metal or metal alloy (e.g., a nickel-titanium alloy or “nitinol”), a plastic or thermoplastic material, a composite material, or any combination thereof. The end effector 204 can articulate in two planes simultaneously, which requires the knife rod 416 to be flexible enough to traverse the articulation joints while still applying adequate cut force. The knife rod 416 may also comprise a braided cable construction of a metal (e.g., stainless steel, tungsten, etc.), or any of the aforementioned materials, and such braided cable may be radially constrained to support axial loads.

Upon firing the end effector 204 with the jaws 210, 212 closed, the knife rod 416 is moved (urged) distally which correspondingly moves the knife 502 to an extended position. After firing is complete, the knife rod 416 is retracted proximally, which correspondingly pulls the knife 502 proximally and back to the stowed position at the knife housing 506 (FIG. 5) until it is desired to again fire the end effector 204. Ensuring the knife 502 is adequately coupled to the knife rod 416 helps ensure that the knife 502 does not prematurely fail or otherwise separate from the knife rod 416.

According to embodiments of the present disclosure, and as described in more detail below, the knife 502 may be operatively coupled to the distal end of the knife rod 416 at one or more retention plateaus defined on one or both surface sides of the knife 502. The retention plateaus help assume any axial loading resulting from operation of the knife 502, including increasing tensile strength and blade retention force.

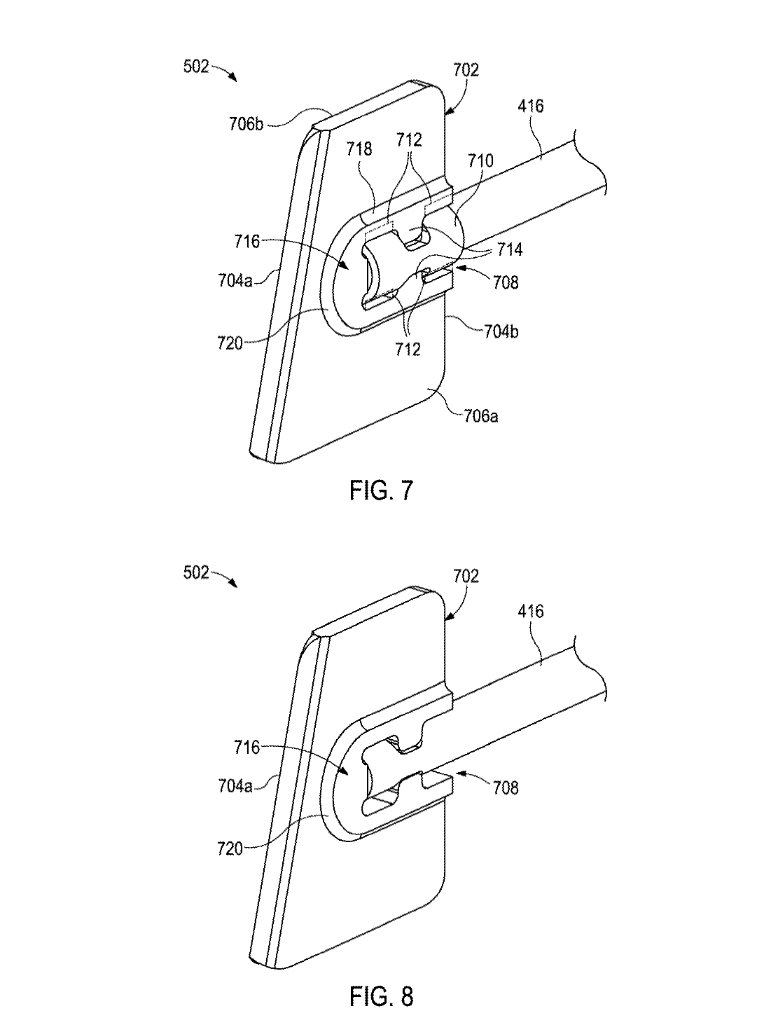

FIG. 7 is an enlarged, isometric view of the knife 502, in accordance with the principles of the present disclosure. As illustrated, the knife 502 provides a generally flat body 702 having a first or “leading” end 704a, a second or “trailing” end 704b, and opposing side surfaces 706a and 706b (second side surface 706b is occluded) extending between the leading and trailing ends 704a,b. The leading end 704a provides the blade portion of the knife 502 configured to cut through tissue, and the knife rod 416 may be attached to the knife 502 at the trailing end 704b. The knife 502 may be made from a variety of rigid materials including, but not limited to, stainless steel, nitinol, titanium, ceramic, or tungsten.

The knife rod 416 is operatively coupled to the knife 502 such that longitudinal movement (e.g., distal or proximal) of the knife rod 416 will correspondingly move the knife 502 in the same direction. To operatively couple the knife 502 to the knife rod 416, the knife 502 may define a central slot 708 sized to receive the distal end of the knife rod 416. More specifically, the central slot 708 may provide an interface in which the distal end of the knife rod 416 may be mated to the knife 502. The central slot 708 extends from the trailing end 704b and penetrates distally into the body 702 towards the leading end 704a.

In some embodiments, the knife 502 may be directly coupled to the knife rod 416 at the central slot 708. In such embodiments, the knife 502 may be welded to the knife rod 416 at the central slot 708. Moreover, in such embodiments the knife 502 and the knife rod 416 may be made of the same material such as, but not limited to, stainless steel or nitinol. Accordingly, in at least one embodiment, it is contemplated herein that both the knife rod 416 and the knife 502 be formed of nitinol, for example, and the knife 502 may be directly attached to the knife rod 416 via a nitinol-to-nitinol weld or coupling. This may prove advantageous in enabling direct welding between the components to form an integrally formed component.

In other embodiments, however, the knife 502 may be operatively coupled to the knife rod 416 at the central slot 708 using an intermediate retention feature 710 attached to the distal end of the knife rod 416. The intermediate retention feature 710 may comprise, but is not limited to, a crimped engagement, a welded interface on an outer surface of the knife rod 416, an adhesive attachment, an interference or shrink fit, an overmold (e.g., a shaped block of material or a support block), one or more mechanical fasteners, or any combination thereof. In at least one embodiment, the intermediate retention feature 710 may comprise a metal tube or “ferrule” crimped or press fit to the distal end of the knife rod 416, and the outer surface of the ferrule may be welded to the knife 502 at one or more weld locations 712 (shown in dashed lines).

In some embodiments, the central slot 708 may define or otherwise provide one or more notches 714 that protrude or extend into the central slot 708. The notches 714 may be formed via grinding (or a grinding process), but could alternatively be formed through other processes, such as laser cutting, wire electrical discharge machining (EDM), milling, chemical etching, water jetting, stamping, or the like. In embodiments that include the intermediate retention feature 710, corresponding notches may be defined in the intermediate retention feature 710 to correspond with and receive the notches 714. In some embodiments, the notches 714 may extend into the material of the knife rod 416, but stop short of the centerline of the knife rod 416. The notches 714 may help increase the mating surface area between the knife 502 and the knife rod 416 (or the intermediate retention feature 710). The notches 714 may further aid in locating the distal end of the knife rod 416 within the central slot 708 prior to operatively coupling the knife rod 416 within the knife 502.

In the illustrated embodiment, two notches 714 are shown axially aligned with each other, which may provide stabilizing forces equally on the top and bottom of the knife rod 416 and prevent the knife rod 416 from backing out from the knife 502 prior to operative coupling. In other embodiments, as described in more detail below, it is contemplated herein to include a single notch 714 or multiple notches 714 that are axially offset from each other.

As illustrated, one or more retention plateaus 716 (one shown) may be defined or otherwise provided on one or both of the side surfaces 706a,b. While the retention plateau 716 (alternately referred to as “raised boss”) is shown in FIG. 7 on the first side surface 706a, it is contemplated herein that the retention plateau 716 may alternatively be provided on the second side surface 706a, or separate retention plateaus 716 may alternatively be provided on both side surfaces 706a,b. In some embodiments, as illustrated, the retention plateau 716 may circumscribe (e.g., extend about) the central slot 708 such that the distal end of the knife rod 416 (and the intermediate retention feature 710) is contained within the central slot 708 and circumscribed by the retention plateau 716.

The retention plateau 716 may comprise a portion of the knife 502 that exhibits an increased material thickness of the knife 502 as compared to adjacent portions of the side surface 706a. Accordingly, the retention plateau 716 may project laterally from the side surface 706a of the knife 502 to provide increased thickness across a full profile of the retention plateau 716. The increased thickness of the retention plateau 716 may add or maximize a retention force between the knife rod 416 and the knife 502, and the maximized retention force may increase a strength of the connection between the knife rod 416 and the knife 502, such that failures at the connection (coupling) may be reduced.

The retention plateau 716 may be formed or fabricated via a variety of processes that result in the raised shape of the retention plateau 716 projecting from the side surface 706a of the knife 502. Example processes include, but are not limited to, photo-chemical machining, etching, machining, grinding, hot/cold stamping or forming, metal injection molding, electric chemical machining (ECM), electric discharge machining (EDM), or any combination thereof.

In some embodiments, the retention plateau 716 may include or otherwise define a transition surface 718 extending between the side surface 706a and the retention plateau 716. The transition surface 718 may provide a rounded, beveled, chamfered, or otherwise smoothed transition between the raised surface of the retention plateau 716 and the side surface 706a. A smooth transition of the transition surface 718 may prove advantageous in avoiding the inclusion of sharp or jagged surfaces on the retention plateau 716 that may inadvertently snag or cut tissue during operation.

In at least one embodiment, as illustrated, the retention plateau 716 may include or otherwise define a leading edge 720 forming part of the transition surface 718 and arranged at the distal end of the retention plateau 716, but terminating prior to the leading end 704a of the knife 502. The shape of the leading edge 720 can affect the overall performance and efficiency of the knife 502 moving through tissue, as well as the guidance and retention of the knife 502 within the knife slot 508 (FIG. 5). As such, the leading edge 720 may exhibit a smooth, beveled, arcuate, curved, or rounded shape that reduces the probability that the retention plateau 716 will interfere with the cutting operation of the knife 502. In the illustrated embodiment, for example, the leading edge 720 is in the general form of a bullnose, but could alternatively exhibit other shapes, without departing from the scope of the disclosure.

FIG. 8 is another enlarged, isometric view of the knife 502, according to one or more additional embodiments of the present disclosure. In the illustrated embodiment, the intermediate retention feature 710 (FIG. 7) is omitted, such that the knife rod 416 may be received within the central slot 708 and directly coupled to the body 702 of the knife 502 without any interposing components. In such embodiments, the knife 502 and the knife rod 416 may be made of the same material, such as nitinol. In some embodiments, the internal surfaces of the central slot 708 may be defined with smaller tolerances between the knife rod 416 and the body 702 of the knife 502, such that the knife rod 416 and the knife 502 may be directly coupled via welding (e.g., the welds 712 of FIG. 7), an adhesive, or the like.

The knife 502 of FIG. 8 may also define or provide an alternate geometry for the retention plateau 716. As illustrated, the retention plateau 716 may circumscribe the central slot 708. The leading edge 720 of the retention plateau 716, however, can exhibit an angularly tapering or conical geometry that extends towards the leading end 704a of the knife 502. The angularly tapering leading edge 720 may help guide cut tissue around the knife 502 and away from the knife rod 416 during operation. The angularly tapering leading edge 720 may further aid in guiding the knife 502 through the knife slot 508 (FIG. 5), such that the knife 502 may be centered and translated therein.

It should be noted that while the disclosed embodiments illustrate only the bullnose and angularly tapering shapes of the leading edge 720, those skilled in the art will readily appreciate that a variety of shapes may be chosen for the leading edge 720, without departing from the scope of the present disclosure.

In some embodiments, the retention plateau 716 of FIG. 8 may be smaller than the retention plateau 716 of FIG. 7, thereby resulting in a smaller profile for the knife 502 of FIG. 8. The reduced overall profile of the knife 502 may provide less surface area protruding from the side surface 706a of the knife 502, and is therefore less prone to catch or contact portions of the knife slot 508 (FIG. 5) provided in the jaws 210, 212 (FIGS. 2 and 4). More particularly, the reduced profile of the retention plateau 716 of FIG. 8 may allow the knife 502 to navigate a curved knife slot 508 more easily and without significant frictional loading, including significant reduction in frictional drag when passing through tissue.

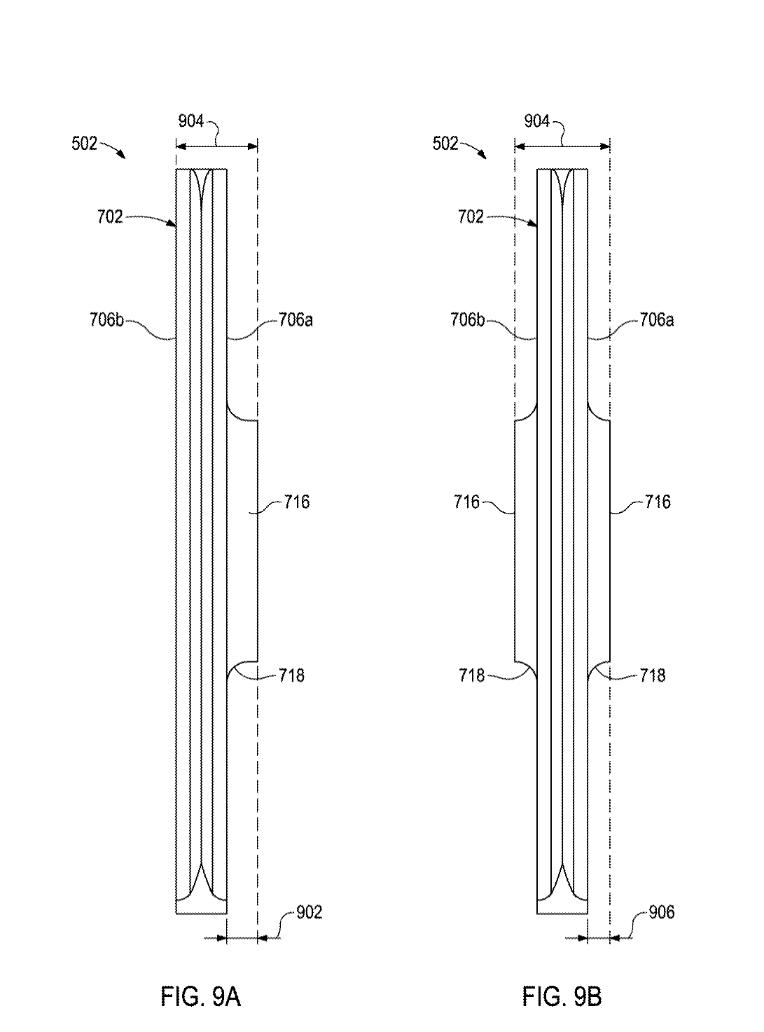

FIGS. 9A and 9B are enlarged front (end) views of the knife 502, according to various embodiments of the present disclosure. As illustrated, the knife 502 provides the generally flat body 702 with the opposing side surfaces 706a,b.

Referring first to FIG. 9A, the knife 502 provides a single retention plateau 716 protruding from the first side surface 706a of the knife 502, and the retention plateau 716 exhibits a first plateau thickness 902. In some embodiments, the retention plateau 716 may enable the knife rod 416 (FIGS. 7 and 8) to be positioned laterally offset from the knife 502, such that the centerline of the knife rod 416 and the centerline of the knife 502 may be misaligned. This misalignment may be useful in certain surgical operations, wherein an offset cut or complex insertion angle may be desirable.

With the single retention plateau 716, the body 702 and the retention plateau 716 may cooperatively exhibit a total thickness 904 large enough such that the knife rod 416 (FIGS. 7 and 8) may be fully seated (located) within the dimension of the total thickness 904 without laterally protruding from the central slot 708 (FIGS. 7 and 8) after mating.

Referring now to FIG. 9B, the knife 502 provides two (dual) retention plateaus 716 protruding from each side surface 706a,b. The dual retention plateaus 716 may enable the positioning of the knife rod 416 (FIGS. 7 and 8) at a center alignment with the knife 502, such that cutting of tissue may be performed centered upon the knife rod 416. In some embodiments, the dual retention plateaus 716 may each define a second plateau thickness 906 that may be less than the first plateau thickness 902 of FIG. 9A. In these embodiments, the combined thickness of the knife 502 and the dual retention plateaus 716 may yield the same total thickness 904 as the knife 502 and single retention plateau 716 of FIG. 9A. As such, the knife rod 416 may be retained within the knife 502 in either an offset or a centrally-aligned manner without affecting the retention force provided by the total thickness 904. In further embodiments, however, the total thickness 904 may vary between the embodiments of FIGS. 9A and 9B without departing from the scope of the present disclosure.

In both FIGS. 9A-9B, a shape of the transition surface 718 may be better seen between the single retention plateau 716 or dual retention plateaus 716 and the side surfaces 706a,b of the knife 502. The transition surface 718 may be seen as a rounded transition that spans the first plateau thickness 902 or second plateau thickness 906, and may be accordingly shaped to provide desirable smoothness at this transition. In some embodiments, as disclosed above, the transition surface 718 may be alternatively chamfered, beveled, tapered, or otherwise smoothed to prevent snagging or nicking of tissues, without departing from the scope of the present disclosure.

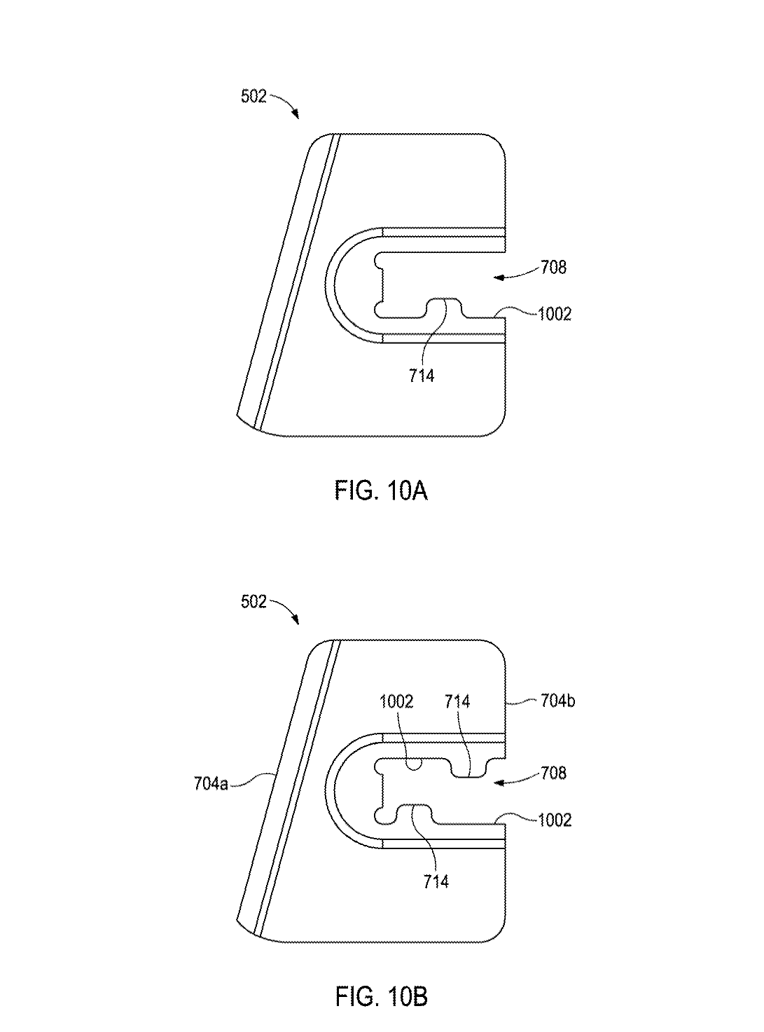

FIGS. 10A and 10B are enlarged side views of alternate examples of the knife 502, according to one or more additional embodiments. More specifically, the knife 502 shown in FIGS. 10A-10B include variations to the notches 714 generally described above. As discussed above, the notches 714 may protrude into the central slot 708 to enable improved location and retention of the knife rod 416 (FIGS. 7 and 8) within the central slot 708. The notches 714 may further reduce slip or displacement within the central slot 708, while providing increased surface area for mating between the knife rod 416 and the knife 502.

Referring first to FIG. 10A, the knife 502 may include a single notch 714 protruding from an internal surface 1002 of the central slot 708. The singular notch 714 is shown roughly centered within the central slot 708, such that a balance of forces may be provided on either side of the singular notch 714. In further embodiments, however, the singular notch 714 may be distally or proximally offset within the central slot 708, without departing from the scope of the present disclosure. Further, while the singular notch 714 is shown as protruding vertically upwards from the internal surface 1002, the singular notch 714 may alternatively protruding vertically downwards from the opposing internal surface of the central slot 708, without departing from the scope of the present disclosure.

Referring now to FIG. 10B, the knife 502 may include two notches 714 axially offset from each other (e.g., axially mis-aligned with each other). More specifically, one notch 714 may be located closer to the leading end 704a within the central slot 708 as compared to the second notch 714, which may be located closer to the trailing end 704b within the central slot 708. The axially offset nature of the notches 714 may enable enhanced mating and slip reduction, as the knife rod 416 (FIGS. 7 and 8) may no longer be able to pivot around a single interference point of axially aligned notches 714, such as shown in FIG. 7. Accordingly, the axially offset notches 714 may reduce the overall play between the knife rod 416 and the knife 502 prior to mating.

While the illustrated embodiment shows one notch 714 protruding vertically upwards from the internal surface 1002 and the second notch 714 protruding vertically downwards from the opposing internal surface 1002, the vertical locations/projections of the notches 714 may be swapped without departing from the scope of the present disclosure. In further embodiments, the locations and numbers of the notches 714 may be optimized to balance mechanical engagement between the knife 502 and the knife rod 416 (FIGS. 7 and 8) and/or intermediate retention feature 710 (FIG. 7). It should also be noted that in both examples of FIGS. 10A-10B, the knife rod 416 (FIGS. 7 and 8) may be correspondingly shaped to mate with the shape of the central slot 708 and any notches 714 therein. As such, the design of the notches 714 may be optimized to further preserve the cross-sectional area of the knife rod 416 (FIGS. 7 and 8) to maintain a desired tensile strength.

### Embodiments disclosed herein include:

A. An end effector for a surgical tool, the end effector comprising opposing first and second jaws, a knife slot defined in one or both of the first and second jaws, and a knife extendable through the knife slot and operatively couplable to a distal end of a knife rod. The knife provides a body having a leading end, a trailing end opposite the leading end, and opposing first and second side surfaces extending between the leading and trailing ends, a central slot defined in the body and extending from the trailing end, the central slot being sized to receive the distal end of the knife rod, and a retention plateau defined on and laterally protruding from the first side surface, wherein the retention plateau circumscribes the central slot.

B. A method of operating a surgical tool, the method comprising positioning the surgical tool adjacent a patient for operation, the surgical tool including a drive housing, an elongate shaft extending from the drive housing, a knife rod extending from the drive housing within the elongate shaft, and an end effector arranged at a distal end of the elongate shaft. The end effector includes opposing first and second jaws, a knife slot defined in one or both of the first and second jaws, and a knife operatively coupled to a distal end of the knife rod at a central slot defined in the knife, the knife including a retention plateau defined on and laterally protruding from a first side surface of the knife, the retention plateau circumscribing the central slot. The method further comprises closing the first and second jaws and thereby grasping onto tissue between the first and second jaws, and actuating the knife rod to advance the knife through the knife slot and thereby cutting the tissue.

C. An end effector for a surgical tool, the end effector comprising opposing first and second jaws, and a knife slot defined in one or both of the first and second jaws, and a knife extendable through the knife slot and operatively coupled to a distal end of a knife rod. The knife provides a body having a leading end, a trailing end opposite the leading end, and opposing first and second side surfaces extending between the leading and trailing ends, a central slot defined in the body and extending from the trailing end, the central slot being sized to receive the distal end of the knife rod, a first retention plateau defined on the first side surface and laterally protruding from the first side surface, a second retention plateau defined on the second side surface and laterally protruding from the second side surface, and a notch protruding into the central slot from an internal surface of the central slot, wherein the first and second retention plateaus each circumscribe the central slot.

Each of embodiments A through C may have one or more of the following additional elements in any combination:

Element 1: wherein retention plateau comprises a first retention plateau and the knife further provides a second retention plateau defined on the second side surface and laterally protruding from the second side surface, wherein the second retention plateau circumscribes the central slot.

Element 2: wherein the body is directly coupled to the knife rod at the central slot.

Element 3: wherein the knife and the knife rod are made of the same material.

Element 4: wherein the body is coupled to an intermediate retention feature attached to the distal end of the knife rod.

Element 5: wherein the knife is welded to an outer surface of the intermediate retention feature.

Element 6: wherein the central slot defines one or more notches protruding from an internal surface of the central slot.

Element 7: wherein the central slot defines first and second notches axially aligned with each other within the central slot.

Element 8: wherein the central slot defines first and second notches axially misaligned with each other within the central slot.

Element 9: wherein the retention plateau is formed from a process selected from the group consisting of photo-chemical machining, etching, machining, grinding, hot/cold stamping or forming, metal injection molding, electric chemical machining (ECM), electric discharge machining (EDM), and any combination thereof.

Element 10: wherein the retention plateau includes a leading edge located at a distal end of the retention plateau.

Element 11: wherein the leading edge exhibits a bullnose shape, an angularly tapering shape, or a conical shape.

Element 12: wherein the knife is secured within the retention plateau such that a leading end of the knife is laterally offset from a centerline of the knife rod. new paragraph...

Element 13: wherein the knife defines a second retention plateau on second side surface of the knife, and wherein the knife is secured within the retention plateaus such that a knife cutting edge is centered with a centerline of the knife rod.

Element 14: wherein the retention plateau includes a leading edge located at a distal end of the retention plateau, and wherein actuating the knife rod further comprises guiding the tissue with the leading edge.

Element 15: further comprising centering and guiding the knife within the knife slot with the retention plateau.

Element 16: wherein the notch is a first notch, and wherein the central slot defines a second notch protruding from the internal surface of the central slot in a direction opposite the first notch.

Element 17: wherein the first and second notches are axially aligned within the central slot.

By way of non-limiting example, exemplary combinations applicable to A through C include: Element 2 with Element 3; Element 4 with Element 5; Element 6 with Element 7; Element 6 with Element 8; Element 10 with Element 11; and Element 16 with Element 7.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. An end effector for a surgical tool, comprising:
opposing first and second jaws;
a knife slot defined in one or both of the first and second jaws; and
a knife extendable through the knife slot and operatively couplable to a distal end of a knife rod, the knife providing:
a body having a leading end, a trailing end opposite the leading end, and opposing first and second side surfaces extending between the leading and trailing ends;
a central slot defined in the body and extending from the trailing end, the central slot being sized to receive the distal end of the knife rod; and
a retention plateau defined on and laterally protruding from the first side surface, wherein the retention plateau circumscribes the central slot.

2. The end effector of claim 1, wherein retention plateau comprises a first retention plateau and the knife further provides a second retention plateau defined on the second side surface and laterally protruding from the second side surface, wherein the second retention plateau circumscribes the central slot.

3. The end effector of claim 1, wherein the body is directly coupled to the knife rod at the central slot.

4. The end effector of claim 3, wherein the knife and the knife rod are made of the same material.

5. The end effector of claim 1, wherein the body is coupled to an intermediate retention feature attached to the distal end of the knife rod.

6. The end effector of claim 5, wherein the knife is welded to an outer surface of the intermediate retention feature.

7. The end effector of claim 1, wherein the central slot defines one or more notches protruding from an internal surface of the central slot.

8. The end effector of claim 7, wherein the central slot defines first and second notches axially aligned with each other within the central slot.

9. The end effector of claim 7, wherein the central slot defines first and second notches axially misaligned with each other within the central slot.

10. The end effector of claim 1, wherein the retention plateau is formed from a process selected from the group consisting of photo-chemical machining, etching, machining, grinding, hot/cold stamping or forming, metal injection molding, electric chemical machining (ECM), electric discharge machining (EDM), and any combination thereof.

11. The end effector of claim 1, wherein the retention plateau includes a leading edge located at a distal end of the retention plateau.

12. The end effector of claim 11, wherein the leading edge exhibits a bullnose shape, an angularly tapering shape, or a conical shape.

13. A method of operating a surgical tool, comprising:
positioning the surgical tool adjacent a patient for operation, the surgical tool including a drive housing, an elongate shaft extending from the drive housing, a knife rod extending from the drive housing within the elongate shaft, and an end effector arranged at a distal end of the elongate shaft, the end effector including:
opposing first and second jaws;
a knife slot defined in one or both of the first and second jaws; and
a knife operatively coupled to a distal end of the knife rod at a central slot defined in the knife, the knife including a retention plateau defined on and laterally protruding from a first side surface of the knife, the retention plateau circumscribing the central slot;
closing the first and second jaws and thereby grasping onto tissue between the first and second jaws; and
actuating the knife rod to advance the knife through the knife slot and thereby cutting the tissue.

14. The method of claim 13, wherein the knife is secured within the retention plateau such that a leading end of the knife is laterally offset from a centerline of the knife rod.

15. The method of claim 13, wherein the knife defines a second retention plateau on second side surface of the knife, and wherein the knife is secured within the retention plateaus such that a knife cutting edge is centered with a centerline of the knife rod.

16. The method of claim 13, wherein the retention plateau includes a leading edge located at a distal end of the retention plateau, and wherein actuating the knife rod further comprises guiding the tissue with the leading edge.

17. The method of claim 13, further comprising centering and guiding the knife within the knife slot with the retention plateau.

18. An end effector for a surgical tool, comprising:
opposing first and second jaws, and a knife slot defined in one or both of the first and second jaws; and
a knife extendable through the knife slot and operatively coupled to a distal end of a knife rod, the knife providing:
a body having a leading end, a trailing end opposite the leading end, and opposing first and second side surfaces extending between the leading and trailing ends;
a central slot defined in the body and extending from the trailing end, the central slot being sized to receive the distal end of the knife rod;
a first retention plateau defined on the first side surface and laterally protruding from the first side surface;
a second retention plateau defined on the second side surface and laterally protruding from the second side surface; and
a notch protruding into the central slot from an internal surface of the central slot,
wherein the first and second retention plateaus each circumscribe the central slot.

19. The end effector of claim 18, wherein the notch is a first notch, and wherein the central slot defines a second notch protruding from the internal surface of the central slot in a direction opposite the first notch.

20. The end effector of claim 19, wherein the first and second notches are axially aligned within the central slot.
